(19) United States
(12) Patent Application Publication
(10) Pub. No.: US 2022/0101960 A1
(43) Pub. Date: Mar. 31, 2022

(54) METHOD AND APPARATUS FOR ANALYSIS OF HISTOPATHOLOGY DATASETS

(71) Applicant: Siemens Healthcare GmbH, Erlangen (DE)

(72) Inventors: Sven KOHLE, Erlangen (DE); Svenja LIPPOK, Uttenreuth (DE); Volker SCHALLER, Erlangen (DE)

(73) Assignee: Siemens Healthcare GmbH, Erlangen (DE)

(21) Appl. No.: 17/477,849

(22) Filed: Sep. 17, 2021

(30) Foreign Application Priority Data
Sep. 28, 2020 (DE) 10 2020 212 189.3

(51) Int. Cl. G16H 10/40; G16H 70/60; G16H 50/20

(52) U.S. Cl. CPC G16H 10/40; G16H 50/20; G16H 70/60

(57) ABSTRACT

Methods and apparatuses are provided for identifying possible further (histo)pathological examinations from already existing histopathological examination results with a computer-implemented and automated analysis. The already existing histopathological examination results in this case are available in the form of a histopathology dataset. The histopathology dataset has one or more first histopathology slide images, which each show a first tissue slide prepared from a tissue sample of a patient and stained with a first histopathological staining. Via an analysis algorithm examination information is established based on the histopathology dataset, with the examination information having a specification for carrying out a further (histo)pathological examination of the patient, in particular by a user making an appraisal.

### PRIORITY STATEMENT

The present application hereby claims priority under 35 U.S.C. § 119 to German patent application number DE102020212189.3 filed Sep. 28, 2020, the entire contents of which are hereby incorporated herein by reference.

### FIELD

Example embodiments of the invention generally relate to methods and apparatuses for analysis of histopathology datasets. In particular, example embodiments of the present invention relate to methods and apparatuses for analysis of histopathology datasets for deriving information for carrying out further histopathological examinations.

### BACKGROUND

The analysis of tissue samples with methods of histopathology is a central element in cancer diagnostics. In such methods tissue samples are taken from a patient from a region of the body in which there may possibly be a pathological change present. Typically a number of sections, which are then cut into micrometer-thick slices known as tissue slides, are cut out of a tissue sample. These sections are usually punched out of the tissue samples and are therefore also known as “punch biopsies”. Another expression for them is “blocks”. To enable possible tissue changes to be better recognized or even quantified at all, the tissue slides are stained with histopathological stainings. The analysis of the stained tissue slides under the microscope by a pathologist then allows information to be provided about possible pathological changes to the fine tissue structure of the tissue being examined.

Histopathological examinations are very labor-intensive. As well as the taking of the tissue samples as such, they require the preparation of the tissue slides, including the cutting, fixing and staining of the tissue slides. In such cases it should be noted that for each tissue sample a plurality of sections and tissue slides must typically be prepared and analyzed.

To relieve the load on the medical personnel, these workflows have therefore in recent decades been ever more automated and digitized. Thus in modern laboratories computer-controlled automatic preparation and staining equipment is often already used. Moreover the stained tissue slides are mostly digitized nowadays for further use. Specialized scanners, known as whole slide scanners, are used for this. The image recorded by the scanners is also referred as a whole slide image. The histopathology image data contained in this way is then viewed and analyzed by a pathologist at a digital diagnostic station.

One problem in this workflow is deciding on suitable histopathological stainings and the tissue slides to be used for them. There are a plurality of different histological stainings, which have been developed over the course of the last 120 years. In first place is mostly hematoxylin-eosin staining (H&E staining) as a routine and overview staining. Going beyond this, further stainings are also called special stainings. Examples of these are kongorot, trichrome stainings auramin O and many others besides. In addition immunohistochemical stainings can also be used as special stainings, with which proteins and other structures can be made visible with the aid of marked antibodies. Examples of these are Ki67 as cell proliferation markers, Her2 immuno stainings as specific markers for breast cancer, CD8 immuno stainings for marking of T cells, or PD-L1 immuno stainings as predictive markers for the success of immunotherapies.

In practice an H&E staining of a few tissue slides is mostly created first of all. These are then examined by the pathologist. Building on this, the pathologist selects further special stainings and associated tissue slides for further examinations of the tissue sample, based upon which they then define further steps and finally create a further appraisal for forwarding to the doctor carrying out the treatment.

The use of special stainings in this case is not only more expensive than the use of the H&E overview staining, but can also be associated with a significant outlay in time. The selection of unsuitable histopathological stainings and/or tissue slides can therefore lead to a significant delay in deciding on the therapy and to additional financial stresses in the clinical workflow. Because of the enormous significance of histopathological appraisals for the treatment of a patient, the selection of unfavorable parameters for a further examination of a tissue sample can further bring about serious negative consequences for the prognosis of the patient. In such cases susceptibility to errors increases with growing specificity of the further stainings.

### SUMMARY

The inventors have discovered that this is all to be seen against the background of the ongoing automation of the upstream processes. On the one hand, the inventors have discovered that this leads to an ever-increasing workload for the pathologist. On the other hand, the inventors have discovered that because of the constantly increasing volumes of data, it becomes ever more difficult for the individual to include all available information and take it into account appropriately in the definition of the parameters for a further histopathological analysis.

At least one embodiment of the present invention provides methods and/or apparatuses that support a user in their decision about carrying out a further histopathological examination starting from an initial histopathological examination.

In accordance with embodiments of the invention, the embodiments are directed to a method, an apparatus, a computer program product or a computer-readable memory medium. Advantageous developments are specified in the claims.

The inventive way is described below both with regard to the embodiments regarding apparatuses and also with regard to the regarding the method. Features, advantages or alternate forms of embodiment mentioned here are likewise to be transferred to the other subject matter and vice versa. In other words the physical claims (which are directed to an apparatus for example) can also be developed with the features that are described or claimed in conjunction with a method. The corresponding functional features of the method are embodied in such cases by corresponding physical modules.

The inventive way is described below both with regard to the embodiments both with regard to methods and apparatuses for establishing information relating to a further histopathological examination and also with regard to methods and apparatuses for adaptation of trained functions. Features and alternate forms of embodiment of data structures and/or functions for methods and apparatuses for determination can be transferred here to similar data structures and/or functions for methods and apparatuses for adaptation. Similar data structures here can be identified in particular by the use of the prefix “training”. Furthermore the trained functions used in methods and apparatuses for establishing information relating to a further histopathological examination can have been adapted and/or provided in particular by methods and apparatuses for adaptation of trained functions.

In accordance with one form of embodiment of the invention, a computer-implemented method for establishing or providing examination information is provided. The examination information relates to a further examination of the patient or of, in particular medical, data that is associated with the patient (patient data). In particular the examination information relates to a further pathological and/or histopathological examination (known for short as (histo)pathological examination) of the patient or of the patient data, with the examination in particular being able to be based on the tissue sample of the patient. The method has a number of steps. One step is directed to the provision of a histopathology dataset. The histopathology dataset has one or more first histopathology slide images, with the first histopathology slide images each showing a tissue slide prepared from a tissue sample of a patient and stained with a first histopathological staining. The tissue slides stained with the first histopathological staining are also called first tissue slides below. A further step is directed to establishing the examination information. The information is established in this case by evaluation of the histopathology dataset or, based on the histopathology dataset, via an analysis algorithm. The examination information in this case has a specification for carrying out one or more (histo)pathological examinations of the patient, with the examination in particular being able to be based on the tissue sample of the patient. The (histo)pathological examinations in this case are carried out and/or assessed in particular by a user making the appraisals. A further step is directed to the provision of the examination information.

In accordance with one embodiment, a system for establishing examination information relating to a further histopathological examination is provided. The system has an interface and a controller. The interface is embodied to receive a histopathology dataset, with the histopathology dataset having one or more first histopathology slide images, which each show a tissue slide prepared from a tissue sample of a patient and stained with a first histopathological staining. The controller is embodied, based on the histopathology dataset, to establish examination information relating to a further histopathological examination, with the examination information having at least one specification for creation of one or more second histopathology slide images from the tissue sample, in particular for further examination by a user making the appraisal, with the one or more second histopathology slide images being different from the one or more first histopathology slide images. The controller is further embodied for provision of the examination information.

In a further embodiment, the invention relates to a computer program product, which comprises a program and is able to be loaded directly into a memory of a programmable controller, and has program code/segments, e.g. libraries and auxiliary functions, for carrying out a method for provision of examination information relating to a further histopathological examination, in particular in accordance with at least one of the aforementioned embodiments, when the computer program product is executed.

In a further embodiment, the invention further relates to a computer-readable memory medium, on which readable and executable program sections are stored for carrying out all steps of a method for provision of examination information relating to a further histopathological examination, in particular in accordance with at least one of the aforementioned embodiments, when the computer program product is executed by the controller.

In a further embodiment, the invention further relates to a computer-implemented method for provision of examination information relating to a further pathological examination, comprising:

provisioning a histopathology dataset, including one or more first histopathology slide images, each first histopathology slide image of the first histopathology slide images showing a first tissue slide prepared from a tissue sample of a patient and stained with a first histopathological staining;

establishing, using an analysis algorithm based on the histopathology dataset, examination information relating to a further pathological examination, the examination information including a specification for carrying out a further pathological examination of the patient; and provisioning the examination information established.

In a further embodiment, the invention further relates to a system for establishment of examination information relating to a further pathological examination, comprising:

an interface embodied to receive a histopathology dataset, the histopathology dataset including one or more first histopathology slide images, each first histopathology slide image of the first histopathology slide images showing a first tissue slide prepared from a tissue sample of a patient and stained with a first histopathological staining; and

a controller embodied to
establish, based on the histopathology dataset, examination information relating to a further pathological examination, the examination information including at least one specification for carrying out a further pathological examination, and
provide the examination information established.

In a further embodiment, the invention further relates to a non-transitory computer program product, storing a program, directly loadable into a memory of a programmable processing unit of a controller, including program code for carrying out the method of an embodiment when the program is executed in the controller.

In a further embodiment, the invention further relates to a non-transitory computer-readable memory medium, storing readable and executable program sections for carrying out the method of an embodiment when the program sections are executed by a controller.

### BRIEF DESCRIPTION OF THE DRAWINGS

Further special aspects and advantages of the invention are evident from the explanations of example embodiments given below with the aid of schematic diagrams. Modifications mentioned in this context can be combined in each case with one another in order to embody new forms of embodiment. In different figures the same reference characters are used for the same features.

In the figures:

FIG. 1 shows a schematic diagram of a form of embodiment of a system for provision of examination information for a further histopathological examination,

FIG. 2 shows a flow diagram of a method for provision of examination information for a further histopathological examination in accordance with a form of embodiment,

FIG. 3 shows a flow diagram of a method for provision of examination information for a further histopathological examination in accordance with a form of embodiment,

FIG. 4 shows a flow diagram of a method for provision of examination information for a further histopathological examination in accordance with a form of embodiment,

FIG. 5 shows a flow diagram of a method for provision of examination information for a further histopathological examination in accordance with a form of embodiment,

FIG. 6, shows a schematic diagram of a form of embodiment of a system for adaptation of an analysis algorithm that is suitable for establishing examination information of a further histopathological examination, and

FIG. 7 shows a flow diagram of a method for adaptation of an analysis algorithm for improvement of a visualization image of a three-dimensional object in accordance with a form of embodiment.

### DETAILED DESCRIPTION OF THE EXAMPLE EMBODIMENTS

The drawings are to be regarded as being schematic representations and elements illustrated in the drawings are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

Various example embodiments will now be described more fully with reference to the accompanying drawings in which only some example embodiments are shown. Specific structural and functional details disclosed herein are merely representative for purposes of describing example embodiments. Example embodiments, however, may be embodied in various different forms, and should not be construed as being limited to only the illustrated embodiments. Rather, the illustrated embodiments are provided as examples so that this disclosure will be thorough and complete, and will fully convey the concepts of this disclosure to those skilled in the art. Accordingly, known processes, elements, and techniques, may not be described with respect to some example embodiments. Unless otherwise noted, like reference characters denote like elements throughout the attached drawings and written description, and thus descriptions will not be repeated. At least one embodiment of the present invention, however, may be embodied in many alternate forms and should not be construed as limited to only the example embodiments set forth herein.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

When an element is referred to as being “on,” “connected to,” “coupled to,” or “adjacent to,” another element, the element may be directly on, connected to, coupled to, or adjacent to, the other element, or one or more other intervening elements may be present. In contrast, when an element is referred to as being “directly on,” “directly connected to,” “directly coupled to,” or “immediately adjacent to,” another element there are no intervening elements present.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Before discussing example embodiments in more detail, it is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments of the present invention. This invention may, however, be embodied in many alternate forms and should not be construed as limited to only the embodiments set forth herein.

Units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one embodiment of the invention relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

In accordance with one form of embodiment of the invention, a computer-implemented method for establishing or providing examination information is provided. The examination information relates to a further examination of the patient or of, in particular medical, data that is associated with the patient (patient data). In particular the examination information relates to a further pathological and/or histopathological examination (known for short as (histo)pathological examination) of the patient or of the patient data, with the examination in particular being able to be based on the tissue sample of the patient. The method has a number of steps. One step is directed to the provision of a histopathology dataset. The histopathology dataset has one or more first histopathology slide images, with the first histopathology slide images each showing a tissue slide prepared from a tissue sample of a patient and stained with a first histopathological staining. The tissue slides stained with the first histopathological staining are also called first tissue slides below. A further step is directed to establishing the examination information. The information is established in this case by evaluation of the histopathology dataset or, based on the histopathology dataset, via an analysis algorithm. The examination information in this case has a specification for carrying out one or more (histo)pathological examinations of the patient, with the examination in particular being able to be based on the tissue sample of the patient. The (histo)pathological examinations in this case are carried out and/or assessed in particular by a user making the appraisals. A further step is directed to the provision of the examination information.

The histopathology dataset is a dataset that can have image data and non-image data. As image data the histopathology dataset can in particular have one or more histopathology slide images. Histopathology slide images can in particular be two-dimensional pixel images. The histopathology slide images each map a tissue slide that has been prepared from a tissue sample of the patient. All histopathology slide images contained in a histopathology dataset can in particular belong to the same tissue sample, i.e. all histopathology slide images contained in a histopathology dataset show tissue slides that were prepared from the same tissue sample of the patient.

The preparation of tissue slides from a tissue sample can comprise the cutting out of a section (also called a ‘punch biopsy’ or a ‘block’) from the tissue sample (with a punch tool for example), with the section being cut into micrometer-thick slices, the tissue slides. The preparation of the tissue slides further comprises the staining of the tissue slides with a histopathological staining. The staining in this case can serve to highlight different structures in the tissue slide, such as e.g. cell walls or cell nuclei or to check a medical indication, such as e.g. a cell proliferation level. In this process different histopathological stainings can be used for different issues. The tissue slides shown in the one or more first histopathology slide images have been stained with a first histopathological staining. As well as the first histopathology slide images, the histopathology datasets can also have further histopathology slide images that have been stained with a histopathological staining other than the first histopathological staining.

For creation of the one or more first histopathology slide images the tissue slides stained with the first histopathological staining can be or can have been digitalized or scanned. To this end the first tissue slides are imaged with a suitable digitalization station, such as for example what is known as a whole slide scanner, which preferably scans the entire tissue slide mounted on the object carrier and converts it into a pixel image. In order to obtain the staining effect through the histopathological staining, the pixel images are preferably color pixel images. Since in the diagnosis both the overall impression of the tissue and also the finely resolved cell structure is of significance, the histopathology slide images typically have a very high pixel resolution. The histopathology slide images can subsequently be digitally processed and in particular archived in a suitable database. Microscopically the histopathology slide images show the fine tissue structure of the tissue sample and in particular the cell structure or the cells contained in the tissue sample. On greater length scales the histopathology slide images show superordinate tissue structures and features, such as e.g. the density and cell morphologically contiguous regions, such as e.g. tumor regions or stroma regions.

As well as image data the histopathology datasets can also contain non-image data or metadata, in which for example the point in time at which the tissue sample was taken, a patient identifier, patient information (another name for this is ‘personal data of the patient’), such as age and gender, a histopathological staining used, a pathological appraisal and an anatomical target region from which the tissue sample has been taken, and more besides can be stored. As an alternative or in addition such metadata can be held separately from the histopathology datasets in the database archiving the histopathology datasets or in a database separate therefrom. Such databases can for example be part of one or more medical information systems, such as for example Hospital Information Systems (HIS), Radiology Information Systems (RIS), Laboratory Information Systems (LIS), Cardiovascular Information Systems (CVIS) and/or Picture Archiving and Communicating Systems (PACS).

‘Provision’ with regard to the histopathology datasets can mean that the datasets are able to be retrieved or are retrieved from a corresponding database in which they are archived, and/or are loaded or are able to be loaded into a processing unit in order for the histopathology image datasets to be subject to one or more processing steps in the data processing unit.

The establishing of the examination information can in particular comprise an evaluation of the histopathology dataset. The establishing of the examination information can further comprise evaluation of the histopathology dataset or of medical data assigned to the patient outside of the histopathology dataset. The establishing of the examination information can further comprise an evaluation of the first histopathology slide images. The establishing of the examination information can further comprise an evaluation of any non-image data or metadata contained in the histopathology dataset. The aforementioned evaluation steps can be carried out in particular by the (correspondingly embodied) analysis algorithm.

The analysis algorithm can be construed in particular as a computer program product, which is embodied for determination of examination information by analysis of the information contained in the histopathology dataset. To this end the analysis algorithm is applied to the histopathology dataset, or the histopathology dataset is entered into the analysis algorithm. The analysis algorithm can have program elements in the form of one or more Instructions for a processor for determination of the examination information. The analysis algorithm can be embodied in particular, for establishing of the examination information, to evaluate the one or more first histopathology slide images. As well as this the analysis algorithm can be embodied to evaluate medical data assigned to the first histopathology slide images, which exists separately from the histopathology dataset.

The analysis algorithm for example can be provided by being kept in a memory facility or loaded into a main memory of a suitable data processing facility or by being generally made available for use.

The examination information provided by the analysis algorithm on this basis has a specification or information that, based on the histopathology dataset and in particular based on first histopathology slide images already present (and also where necessary based on assigned medical data), is directed to the carrying out of a further (histo)pathological examination or is directed to analysis of the patient or of patient data, in order in particular to form and/or to create a final diagnosis in the pathology workflow. The further examination as such is preferably carried out in this case essentially by a user making the appraisal. The user in this case can in particular be a pathologist or in general a clinician or a doctor. The examination information can contain specifications about which steps for further examination of the patient are indicated, recommended, mandatory, expedient and/or probable. The further (histo)pathological examination thus relates to an examination of the patient or of patient data associated with the patient not yet carried out but where necessary still to be carried out given the state of things.

The examination information can have a specification in respect of a further histopathological examination of the tissue sample of the patient. The examination information can further have a specification in respect of obtaining further patient data. The examination information can further comprise a specification in respect of a molecular pathological analysis of the tissue sample or of another tissue sample of the patient. This specification in respect of a molecular pathological analysis can for example comprise one or more parameters of a molecular pathological analysis to be carried out. The examination information can further comprise a specification about whether a further tissue sample of the patient is to be taken and, optionally about which parameters are to be taken into account for the corresponding tissue removal. The examination information can further comprise a specification about whether a consultation with a further pathology expert or with an expert outside the pathology workflow is indicated.

The provision of the examination information can comprise a provision of the examination information for any given further use. For example the examination information can be transferred to a further algorithm or a system for bringing about the creation of histopathology slide images. The examination information can further be provided for archiving in a database. Furthermore the examination information can be provided to a user for their attention.

Through the provision of the examination information a statement is automatically made available about which further examination, diagnosis and analysis steps would be suitable, starting from the histopathology dataset, for the further analysis of the patient to form and firm up a (histo)pathological appraisal. This puts valuable information at the user's disposal for planning the further pathological appraisal and to underpin possible medical diagnoses or to invalidate them. This enables not only the further examination of the patient to be designed in a more targeted manner, which saves time and money, but also enables the hit rate for the pathological appraisal to be increased. The provision of the examination information further enables clinical processes thus to be designed more efficiently. Moreover therapy decisions can be taken earlier, which can have a positive effect on the success of therapy. Based on an automated evaluation of digitized measurement data, the histopathology datasets, the inventors have thus created a method that sustainably supports a user in the making of medical diagnosis.

In other words, in accordance with a few embodiments of the invention, methods and apparatuses are provided, through a computer-implemented and automated analysis of already available histopathological examination results, to identify further (histo)pathological examinations. The already available histopathological examination results in this case are available in the form of a histopathology dataset. The histopathology dataset has one or more first histopathology slide images, which each show a first tissue slide prepared from a tissue sample of a patient and stained with a first histopathological staining. Via an analysis algorithm examination information is established, based on the histopathology dataset, with the examination information having a specification for carrying out a further (histo)pathological examination of the patient in particular by a user making the appraisal.

In accordance with one embodiment, the examination information has a specification for creation of one or more second histopathology slide images, with the one or more second histopathology slide images being different from the one or more first histopathology slide images.

The examination information can thus in particular contain a specification, information and/or a suggestion relating to the creation or one or more second histopathology slide images. The second histopathology slide images in this case are different from the first histopathology slide images. Different in this context can in particular mean that the second histopathology slide images are based on different tissue slides and/or different histopathological stainings from the one or more first histopathology slide images. The further examination can then for example comprise an inspection of the second histopathology slide images or a use of one or more evaluation tools by the user. In addition or as an alternative an automatic or semi-automatic analysis of the second histopathology slide images is conceivable.

Through the provision of such examination information a statement is automatically made available as to which second histopathology slide images, starting from the histopathology dataset, would be suitable for the further analysis of the tissue sample of the patient. The user can thereby for example be given assistance in the definition of the parameters for the second histopathology slide images. This puts valuable information at the user's disposal for planning the further pathological appraisal and possible medical diagnoses by ordering further histopathology slide images and for underpinning possible medical diagnoses or invalidating them. This not only enables the ordering of second histopathology slide images be designed in a more targeted manner, which saves time and money, but also enables the hit rate in the pathological appraisal to be increased. The provision of the examination information further enables ordering processes to be initiated and clinical processes thus to be designed more efficiently. Moreover therapy decisions can be taken earlier, which can have a positive effect on the success of therapy.

In accordance with one embodiment, the examination information comprises a specification in respect of one or more further histopathological stainings, wherein the one or more further histopathological stainings are different from the first histopathological staining and are suitable for creation of the second histopathology slide images.

In other words, based on at least the histopathology dataset, one or more second histopathological stainings are thus suggested that could be relevant for a further analysis of the tissue sample of the patient. Depending on the respective case, a wide variety of histopathological stainings can be considered, which each have different implications for a future diagnosis and subsequent therapy. The sheer plurality of possibilities not only often poses problems for less experienced users in the ordering of suitable histopathological stainings. The automated suggestion of histopathological stainings as part of the examination information gives assistance here. The user is thereby supported in obtaining a diagnosis and time-consuming and resource-consuming subsequent measurements can be avoided.

In accordance with one embodiment, the examination information comprises a specification in respect of one or more second tissue slides prepared from the tissue sample, which are different from the first tissue slides and are suitable for creating the second histopathology slide images. In particular the examination information has a specification about a section from the tissue sample to be used in the preparation of the second histopathology slide images.

The inventors have recognized that not all tissue slides able to be prepared from a tissue sample are equally well suited to the creation of second histopathology slide images. In particular it is often desired, for a comparison with the first histopathology slide images, that the second histopathology slide images map one or more tissue slides that have a similar tissue region and/or a similar cell density, e.g. of tumor cells, and tissue structure to the tissue slides mapped in the one or more first histopathology slide images. Consequently it can be advantageous for the tissue slides for first and second histopathology slide images to have been taken from the same punch biopsy or the same block by consecutive cuts and/or to be adjacent. On the other hand it can occur again and again that individual tissue slides, maybe through errors in preparation, are unsuitable for further use. The automated selection of suitable second tissue slides enables the user to be relieved of this work.

To this end the analysis algorithm can in particular be designed in such a way that it analyses the first histopathology slide images by way of methods of image analysis and searches in the available tissue slides of the tissue sample for tissue slides that are as similar as possible. A similarity between tissue slides can in particular comprise a morphological or structural similarity between regions of the tissue slides. For example similar regions can have a similar tissue structure, a similar texture, similar pixels or color values, a similar cell density, a similar cell morphology or cell morphologies, similar patterns and/or further similar features. In addition or as an alternative the histopathology dataset can contain information about possible second tissue slides as assigned medical data.

In accordance with one embodiment, the histopathology dataset comprises a number of first histopathology slide images and the step of establishing includes a step of selecting one or more histopathology slide images, wherein the examination information is created based on the selected histopathology slide images and/or the examination information has a specification for creation of one or more second histopathology slide images appropriate to the selected histopathology slide images.

The automatic selection of histopathology slide images enables especially relevant first histopathology slide images to be identified, starting from which second histopathology slide images can be especially informative.

In accordance with one embodiment, the establishing of the examination information comprises establishing of the respective section of the selected histopathology slide images. The examination information can thus comprise a specification of the respective section of the selected histopathology slide images. The examination information can further comprise a specification about one or more tissue slides adjacent to or in particular consecutive to the selected histopathology slide images.

This enables second histopathology slide images that are as similar as possible to the selected histopathology slide images to be identified, which can make the comparability and thus the appraisal easier. Adjacent or consecutive tissue slides in this case are tissue slides that follow on directly or at a slight distance from one another in a section.

In accordance with one embodiment, the selection of the selected histopathology slide images from the first histopathology slide images comprises a selection based on the image data of the first histopathology slide images and in particular based on one or more structural and/or morphological features extracted from the image data. Such a feature can in particular comprise a proportion of the tumor cells in the respective first histopathology slide image (which can be determined for example based on the first histopathological staining).

In accordance with one embodiment, the analysis algorithm can be embodied for selection from the first histopathology slide images in particular according to the criteria. As an alternative or in addition to an automatic selection, there can also be provision, above and beyond this, for making a selection from the first histopathology slide images through a user input (see below) possible.

In accordance with one embodiment, the examination information for each further histopathological staining comprises a specification of suitable second tissue slides in each case.

Thus a suitable tissue slide can be specified for each histopathological staining. This means that the user is supported in the planning of the appraisal in an even more targeted manner, which arranges the processes in the histopathological appraisal more efficiently and further eliminates sources of errors.

In accordance with one embodiment, the method further has the step of receiving a user entry relating to the creation of the one or more second histopathology slide images, wherein the step of establishing is additionally undertaken based the user entry.

The possibility of taking into account the user entry enables the user to influence the automatically created suggestion for creation of second histopathology slide images and in this way tailor it to their preferences or to differential diagnoses that already exist.

In accordance with one embodiment, the user entry can include a specification relating to one or more second histopathological stainings that are different from the first histopathological staining. The examination information can then in particular have a specification in respect of one or more second tissue slides prepared from the tissue sample, which are different from the first tissue slides and are used for creation of the second histopathology slide images with the one or more second histopathological stainings specified in the user entry.

This enables the user in particular to specify a histopathological staining that appears suitable to them and, starting therefrom, the method determines a suitable second tissue slide. ‘Suitable’ tissue slides for creation of the second histopathology slide images in such cases can in particular be when they are able to be compared well with the first tissue slides or with a subregion selected from them. This not only relieves the user of work but sources of error are also eliminated.

In accordance with one embodiment, the user entry can be a specification relating to a suspected diagnosis of the user, in particular based upon the histopathology dataset and/or of further medical data assigned to the patient.

In other words this enables the examination information thus to be tailored explicitly to an existing hypothesis of the user. The selection of suitable second histopathology slide images enables such hypotheses to be explicitly tested. For this the analysis algorithm can in particular be designed to establish a connection between suspected diagnoses and associated histopathological stainings, which are suitable for supporting and/or refuting the corresponding suspected diagnoses. Such connections can be set up in the analysis algorithm for example in the form one or more ontologies or decision trees. In addition the analysis algorithm can be embodied to evaluate electronic medical textbooks, such as the Thieme® eRef.

In accordance with one embodiment, the histopathology dataset has tissue sample information and the step of establishing is additionally undertaken based on the tissue sample information.

In this case the tissue sample information can have a specification about the location at which the tissue sample was taken, the type and/or location of the punch biopsies or sections taken from the tissue sample and/or the type and/or location of the tissue slides formed. In addition or as an alternative the tissue sample can have information in particular as a specification about the type and/or location of the first tissue slides. In addition the tissue information can contain a specification about the condition of the tissue, e.g. in the form of a macroscopy report.

The taking into account of the tissue sample information enables suitable tissue slides explicitly to be selected for creation of the second histopathology slide images, which can further improve the hit rate of the method and thus the support for the user in the histopathological appraisal.

In accordance with one embodiment, the method further has the step of retrieval of the histopathology dataset or of the medical data assigned to the patient, wherein the step of establishing is additionally undertaken based on the assigned medical data. The assigned medical data is in particular data or datasets separate from the histopathology dataset. In such cases the assigned medical data can in particular have one or more items of laboratory data of the patient, one or more items of radiology data of the patient, one or more, in particular radiological appraisals of the patient, and/or one or more earlier histopathological examinations of the patient. In particular the assigned medical data can be kept in a medical information system having one or more databases. The medical information system can for example be embodied as a Hospital Information System (HIS), Radiology Information System (RIS), Laboratory Information System (LIS), Cardiovascular Information System (CVIS) and/or Picture Archiving and Communicating System (PACS). In particular the assigned medical data can have an Electronic Medical Record or EMS for short) of the patient.

The retrieval can for example comprise an interrogation of the medical information system and/or corresponding databases. For example a code can be extracted for this purpose from the histopathology dataset, which uniquely assigns the medical data to a patient or to a histopathology dataset. Such a code can for example be a patient ID, the name of the patient or a case number. Then, with this code, a search can be made in a medical information system for the assigned medical data. In particular the analysis algorithm can be embodied to retrieve the assigned medical data.

As an alternative or in addition information equivalent to the assigned medical data can also be held in the histopathology dataset itself—for example as metadata and/or non-image data. This information can then be extracted directly from the histopathology dataset.

The taking into account of the assigned medical data enables a differentiated picture of the appraised case to be obtained. For example, through the evaluation of previously known pathological and/or radiological appraisals, possible diseases can be isolated and thereby the examination information firmed up. The taking into account of laboratory and/or radiology data further makes it possible to automatically incorporate information outside the pathological diagnosis process, which relieves the load on the user and improves the decision base. In respect of the evaluation of the assigned medical data the analysis algorithm can be embodied to analyze this data automatically and to extract relevant information. For this the evaluation algorithm can for example have a text analysis module available to it (called a natural language processing module or NLP model for short). Radiology data can further comprise one or more items of radiological image data. The analysis algorithm can accordingly have a module for evaluation of radiological image data.

In accordance with one embodiment, the method further has the step of retrieving patient information, and the step of establishing the examination information is additionally undertaken based on the patient information. An item of patient information can for example comprise one or more of the following items: The age, the gender, one or more previous illnesses, information about a medical history of the patient, information about a medical history of a relative of the patient, and/or information about a patient's lifestyle. The patient information can be contained in the histopathology dataset for example. As an alternative or in addition the patient information can be contained in the additional medical data. Accordingly the retrieval of the patient information can comprise an extraction of the patient information from the histopathology dataset or from the retrieved assigned medical data. In particular the analysis algorithm can be embodied to obtain the patient information from the histopathology dataset and/or from the assigned medical data.

The patient information can deliver pointers to pathological tissue changes. If for example ‘smoker’ is specified for a patient under lifestyles, then this can indicate possible pathological tissue changes and thus further examinations than if this is not the case. The same applies to previous illnesses, the age or the gender of the patient. In this way, in the automatic establishing of the examination information, good account is taken of the circumstances of the individual case, which can lead for example to targeted second histopathology slide images.

In accordance with one embodiment, the method further has a step of determination of a specification relating to the user making the appraisal, wherein the step of establishing is additionally undertaken based on the specification relating to the user making the appraisal.

The specification relating to the user making the appraisal can for example comprise a name of the user making the appraisal, information about one or more histopathological stainings that the user has used in the analysis of earlier histopathology datasets and/or information relating to one or more of the user's earlier histopathological appraisals. The specification relating to the user making the appraisal can for example be contained in the histopathology dataset. As an alternative or in addition the specification relating to the user making the appraisal can be contained in the assigned medical data. Accordingly the determination of the specification relating to the user making the appraisal can comprise the establishing of the specification relating to the user making the appraisal from the histopathology dataset or from the retrieved assigned medical data. In particular the analysis algorithm can be embodied to determine the specification relating to the user making the appraisal from the histopathology dataset and/or from the assigned medical data.

In accordance with one embodiment, the method has a step of identifying one or more relevant medical guidelines for the histopathology dataset for the patient, wherein the step of establishing is additionally undertaken based on the one or more medical guidelines established.

In particular the step of identification can be undertaken based on the user entry, the assigned medical data, the histopathology dataset and/or the first histopathology slide images. In particular the analysis algorithm can be embodied to identify the one or more medical guidelines.

Medical guidelines can specify possible next steps in respect of a further histopathological examination for a possible diagnosis or a case group. A case group can be defined for example in this case by membership of a patient cohort, which for its part can be defined by general demographic conditions, such as for example age, gender, etc., and the presence of a specific type of illness. For example next steps can comprise recommended second histopathological stainings. The possible diagnosis and/or the membership of a case group can be predetermined by the user entry. As an alternative or in addition the possible diagnosis and/or the membership of a case group can be established automatically from the information available, for example by evaluation of the assigned medical, data, e.g. in the form of a previously known radiological appraisal.

In accordance with one embodiment, the method further has a step of selection of one or more similar histopathology datasets from a series of reference histopathology datasets. The selected similar histopathology datasets in this case have a defined similarity with the histopathology dataset, wherein the step of establishing is additionally undertaken based on the similar histopathology datasets.

Further histopathological examinations carried out can be known in advance in each case for the reference histopathology datasets. In particular one or more second histopathological stainings used for the reference histopathology datasets can be known in advance. For example information relating to the further histopathological examinations carried out for the reference histopathology datasets can be stored directly in the reference histopathology datasets, e.g. as metadata. As an alternative or in addition this information can be available as medical data assigned to the reference histopathology datasets. The reference histopathology datasets can basically have the same configuration as the histopathology dataset, and thus have both image data and also non-image data. As image data however, the reference histopathology datasets, as well as one or more first histopathology slide images, can have one or more second histopathology slide images, which have been created by application of one or more second histopathological stainings. The reference histopathology datasets can be stored in a database for example.

In accordance with one embodiment, the selection comprises an extraction of a first feature signature from the histopathology dataset and/or from medical data assigned to the histopathology dataset and an establishing of the similar histopathology dataset from the reference histopathology datasets based on the extracted first feature signature.

Taking the reference histopathology datasets into account enables similar cases to the present case from the past to be identified, in which a further histopathological examination has already been carried out. This can be taken into account in an advantageous manner in establishing other possible further examinations for the present case. In particular in this way for example possible second histopathological stainings can be identified, which have been proven in similar situations.

The first feature signature can have one or more features that have been extracted from the histopathology dataset and/or from the assigned medical data and/or extracted from the user entry or computed from this. The feature signature can have features extracted from the image data of the histopathology dataset and/or from metadata belonging to the histopathology dataset (the assigned medical data). The first feature signature can thus characterize the present case or the patient, in particular in the context of a histopathological appraisal. The features of the first feature signature can be grouped together into a feature vector. In particular the first feature signature can have a feature vector. The features can be morphological and/or structural and/or a texture relating to and/or a pattern relating to features of the first histopathology slide images. In particular the features can comprise a tissue structure or a tissue density. The features can further have a cell density, a cell morphology, a distribution of a histopathological staining, a cell size, a proportion of tumor cells and the like. The features can further comprise one or more categorical variables, such as e.g. the first histopathological staining used, one or more suspected appraisals, a specification of a user making the diagnosis and/or a specification of a doctor making a referral etc. The features can further comprise one or more specifications from the patient information and/or from the tissue information. In particular the analysis algorithm can be embodied to extract the first feature signature. The analysis algorithm can further be embodied, based on the first feature signature, to establish the similar histopathology datasets from the reference histopathology datasets.

The establishing of the similar histopathology datasets can further comprise a comparison of the first feature signature with the second feature signatures of the reference histopathology datasets corresponding to the first feature signature. The second feature signatures preferably have the same configuration as the first feature signature. In particular the second feature signatures have at least one subset of the first feature signature. The comparison of the first and second feature signatures can further comprise a determination of the measure of similarity between the respective second feature signatures and the first feature signature. The similar histopathology datasets can then be selected from the reference histopathology datasets based on the respective measures of similarity. In particular the analysis algorithm can be embodied to carry out the described comparison of the first and second feature signatures.

The comparison and also the determination of the measures of similarity can be based for example on the determination of a distance between the first feature signature and the respective second feature signature, the calculation of a cosine similarity of the first and second feature signatures and/or the calculation of a weighted sum of the difference or the similarity between individual features of the first and second feature signatures. Similar histopathology datasets can in particular be those of the reference histopathology datasets of which the associated measure of similarity is greater than a predetermined or predeterminable threshold.

The establishing of the similar histopathology datasets can further comprise an extraction of the second feature signatures from the reference histopathology datasets. In particular the analysis algorithm can be embodied to extract the second feature signatures from the reference histopathology datasets. As an alternative or in addition the second feature signatures can already be set up.

The use of feature signatures enables an easy-to-implement comparison object to be defined. Moreover the features contained in the feature signatures are based on superordinate observables, which often deliver a good statement about the characteristics of a case.

In accordance with one embodiment the user making the appraisal is informed about the selected similar histopathology datasets via a user interface. In this case for example individual elements of the similar histopathology datasets, such as for example first and/or second histopathology slide images, one or more earlier appraisals, and/or one or more further histopathological examinations carried out in conjunction with the similar histopathology datasets are displayed to the user.

The user making the appraisal is thereby given an overview about similar cases and is supported thereby in the diagnosis of the present case.

In accordance with one embodiment the first histopathological staining is a hematoxylin-eosin staining. The hematoxylin-eosin staining is an overview staining, which represents a good starting point for the determination of any further histopathological examinations and provides first indications of pathological changes of the tissue.

In accordance with one embodiment the step of provision of the examination information comprises an output of the examination information via the user interface to the user making the appraisal.

The user is thereby not only informed about the result of the automatic establishment, but is moreover given the ability to evaluate the result and modify it if necessary.

In accordance with one embodiment, the method further comprises a step of receiving an acknowledgement relating to the examination information. In particular the acknowledgement can come from the user making the appraisal and be received via the user interface. As an alternative the acknowledgement can also be appended to the definition of the further (histo)pathological examination and e.g. only be given when the results of the further (histo)pathological examination carried out based on the examination information are present. The acknowledgement can thus in particular be based on a (histo)pathological examination carried out based on the examination information. This acknowledgement can for example comprise an estimation of whether second histopathology slide images produced in response to the examination information have proved helpful in the clinical trajectory of the patient. Such an estimation for example can be made by a user making the appraisal or also by other clinicians involved, e.g. within the framework of a tumor board session. If such acknowledgements are fed back to the algorithm, this can successively “learn something new” by the algorithm being adapted based on the acknowledgements. As an alternative such estimations can be used in order to create or to adapt medical guidelines that the analysis algorithm in accordance with one embodiment can access.

In accordance with one embodiment, the method further comprises a step of adaptation of the examination information based on the acknowledgement. In accordance with one embodiment the method further comprises a step of provision of the acknowledgement to the analysis algorithm for adaptation of the analysis algorithm.

In other words an ongoing human-machine interaction is realized in this way, in order to optimize the result of the establishment. The user is in this way given the opportunity of influencing the result of the establishment before further steps are initiated. The optional acknowledgement to the analysis algorithm further opens up the possibility of continuously improving the algorithm. In accordance with one embodiment the acknowledgement can also be a confirmation of the examination information by the user making the appraisal. The adaptation of examination information can then simply be its verification.

In accordance with one embodiment, the provision of the examination information comprises entering the examination information into a robot staining system for creating the one or more second histopathology slide images using one or more histopathological stainings, and/or forwarding the examination information to an electronic ordering system.

These steps of adaptation of the examination information can be appended to the user's acknowledgement. The forwarding of the examination information to the robot staining system and/or an electronic ordering system enables steps for creation of one or more second histopathology slide images to be initiated automatically. Through this the load on the user making the appraisal can be reduced further and the diagnosis created more quickly overall.

In accordance with one embodiment, the examination information comprises a confidence specification, which specifies the applicability (or relevance of, or confidence in) the examination information for the patient.

In this way the user making the appraisal can estimate how ‘safe’ the analysis algorithm is with the examination information. The user can thereby recognize which examination information or which parts of the examination information they can accept for further use or where they may still have to adjust them. In particular the confidence specification can comprise a confidence value for each second histopathological staining. In this way the user making the appraisal can select in a targeted manner those second histopathological stainings that are best suited for the present case for a further examination.

In accordance with one embodiment, an explanatory specification is further provided which gives information about how strongly particular parameters derived from the histopathology dataset and/or the assigned medical data have contributed to the examination information. In particular the analysis algorithm can be embodied for determination of the explanatory specification. The explanatory specification can then for example be provided to the user, e.g. via a user interface.

The explanatory specification enables the user to better understand the analysis result and in this way for example counteract any incorrect weightings of individual parameters (e.g. by their acknowledgement).

In accordance with one embodiment, the acknowledgement is in particular an aforementioned user entry relating to the creation of the one or more second histopathology slide images.

In accordance with one embodiment, the analysis algorithm has a trained function.

A trained function generally maps input data to output data. The output data can in particular furthermore depend on one or more parameters of the trained function. The one or more parameters of the trained function can be determined and/or adapted by training. The determination and/or the adaptation of the one parameter or the number of parameters of the trained function can be based in particular on a pair consisting of training input data and associated training output data, wherein the trained function is applied to the training input data to create training mapping data. In particular the determination and/or the adaptation can be based on a comparison of the training mapping data and the training output data. In general a trainable function, i.e. a function with not yet adapted parameters, can be referred to as a trainable function. By training one or more trainable functions optionally contained in the analysis algorithm, the analysis algorithm can be embodied to carry out one or more of the tasks described in conjunction with the analysis algorithm, thus e.g. the establishing of the examination information, the evaluation of the histopathology datasets, the furnishing and evaluation of medical data assigned to the histopathology dataset, the incorporation of medical guidelines, the selection of relevant histopathology slide images from the number of first histopathology slide images that may possibly be present and/or the selection of similar histopathology datasets. If a number of these tasks are realized by a trained function, the analysis algorithm can have a separate trained function for each of these tasks. As an alternative or in addition a trained function can be embodied or trained to handle a number of these tasks through to all tasks.

Other terms for trained function are trained mapping specification, mapping specification with trained parameters, function with trained parameters, algorithm based on artificial intelligence, machine-learning algorithm.

In accordance with one embodiment, the trained function has an electronic classifier.

The electronic classifier is in particular embodied to assign one or more further histopathological examination steps to the histopathology dataset as a result of the properties and features of the histopathology dataset and to output this assignment as examination information. To this end the electronic classifier can for example select from a number of possible further histopathological examination steps those steps that appear especially suited based on the histopathology dataset. For this the electronic classifier can refer back facultatively to cases of comparisons from the past (the reference histopathology datasets) and assign the present histopathology dataset based on the comparison cases. The electronic classifier can further take account of medical guidelines and/or earlier actions of the user making the appraisal.

In accordance with one embodiment, the trained function can have a support vector machine algorithm, a decision tree algorithm, a k-nearest neighbors algorithm, a Bayesian classification algorithm, an, in particular convolutional, neural network and/or combinations hereof.

The inventors have recognized that the aforementioned machine learning schemes are suitable for machine learning for the establishing of the examination information. In particular the convolutional neural network can be embodied as a deep convolutional neural network. The neural network in this case has one or more convolutional layers and one or more deconvolutional layers. In particular the neural network can comprise a pooling layer. The use of convolutional layers and/or deconvolutional layers enables a neural network to be used especially efficiently for image processing.

In accordance with one embodiment, the trained function is in particular embodied, in the step of establishing, to evaluate the one or more first histopathology slide images.

In particular a trained function can be trained. In particular the training of a trained function is carried out based on the training input data and the associated training output data in accordance with a supervised learning technique, wherein the known training input data is entered into the trained function and output data generated by the trained function is compared with the associated training output data. The trained function learns and adapts its parameters for as long as the output data does not sufficiently correspond to the training output data.

In accordance with one embodiment, the trained function can further be embodied so that, based on the acknowledgement of the user, it can continue to be trained by an adaptation of the trained function based on the acknowledgement. This enables continuous learning and thus a constant improvement of the trained function to be achieved.

In particular this type of local improvement and improvement achieved for the local cases can be incorporated into what is known as a master of the trained function, with the master in particular being managed centrally. This principle is called federated learning. In accordance with one embodiment the trained function is provided by a federated learning method.

In accordance with one embodiment, a computer-implemented method for provision of a trained function for automatically establishing examination information relating to a further histopathological examination based on histopathology datasets of a patient is provided. The method has a number of steps. A first step is directed to the provision of a trained function, which is to be (further) adapted for this task. A further step is directed to the provision of a training histopathology dataset, with the training histopathology dataset having at least one histopathology slide image, with the histopathology slide image showing a section of a tissue slide prepared from a tissue sample of a patient and stained with a first histopathological staining. A further step is directed to a provision of verified examination information relating to a further histopathological examination for the training histopathology dataset. A further step is directed to the establishment of training examination information by inputting the training histopathology dataset into the trained function. A further step is directed to a comparison of the verified examination information with the training examination information established. A further step is finally directed to an adaptation of the trained function based upon the comparison.

In accordance with one embodiment, the verified examination information can comprise a second histopathological staining. In accordance with one embodiment the method can further comprise the provision of assigned medical data, one or more medical guidelines, one or more reference histopathology datasets, patient information, user information and/or a user entry to the histopathology dataset, wherein the trained function additionally establishes the examination information based on the one or more medical guidelines, one or more reference histopathology datasets, patient information, user information and/or user entry provided to the medical data assigned to the histopathology dataset.

In accordance with one embodiment, a system for establishing examination information relating to a further histopathological examination is provided. The system has an interface and a controller. The interface is embodied to receive a histopathology dataset, with the histopathology dataset having one or more first histopathology slide images, which each show a tissue slide prepared from a tissue sample of a patient and stained with a first histopathological staining. The controller is embodied, based on the histopathology dataset, to establish examination information relating to a further histopathological examination, with the examination information having at least one specification for creation of one or more second histopathology slide images from the tissue sample, in particular for further examination by a user making the appraisal, with the one or more second histopathology slide images being different from the one or more first histopathology slide images. The controller is further embodied for provision of the examination information.

The controller can be embodied as a central or local processing unit. The processing unit can have one or more processors. The processors can be embodied as a central processing unit (abbreviated to CPU) and/or as a graphics processing unit (abbreviated to GPU). As an alternative the controller can be implemented as a local or Cloud-based processing server.

The interface can generally be embodied for the exchange of data between the controller and further components. The interface can be implemented in the form of one or more individual data interfaces, which can have a hardware and/or software Interface, e.g. a PCI bus, a USB interface, a Firewire interface, a ZigBee or a Bluetooth interface. The interface can further feature an interface of a communication network, wherein the communication network can feature a Local Area Network (LAN), for example an Intranet or a Wide Area Network (WAN). Accordingly the one or more data interfaces can have a LAN interface or a Wireless LAN interface (WLAN or Wi-Fi).

The advantages of the proposed apparatus essentially correspond to the advantages of the proposed method. Features, advantages or alternate forms of embodiment can likewise be transferred to the other claimed subject matter and vice versa.

In a further embodiment, the invention relates to a computer program product, which comprises a program and is able to be loaded directly into a memory of a programmable controller, and has program code/segments, e.g. libraries and auxiliary functions, for carrying out a method for provision of examination information relating to a further histopathological examination, in particular in accordance with at least one of the aforementioned embodiments, when the computer program product is executed.

In a further embodiment, the invention further relates to a computer-readable memory medium, on which readable and executable program sections are stored for carrying out all steps of a method for provision of examination information relating to a further histopathological examination, in particular in accordance with at least one of the aforementioned embodiments, when the computer program product is executed by the controller.

The computer program products in this case can comprise software with a source code that still has to be compiled and linked or only has to be interpreted, or an executable software code, which for execution only has to be loaded into a processing unit. The computer program products enable the methods to be carried out quickly, identically repeatably and robustly. The computer program products are configured so that the processing unit can carry out the inventive method steps. The processing unit in such cases must have the respective prerequisites, such as for example a corresponding main memory, a corresponding processor, a corresponding graphics card or a corresponding logic unit, so that the respective method steps can be carried out efficiently.

The computer program products are stored for example on a computer-readable storage medium or are held on a network or server, from where they can be loaded into the processor of the respective processing unit, which can be directly connected to the processing unit or can be embodied as a part of the processing unit. Furthermore control information of the computer program products can be stored on a computer-readable storage medium. The control information of the computer-readable storage medium can be embodied in such a way that, when the data medium is used in a processing unit, it carries out an embodiment of an inventive method. Examples of a computer-readable storage medium are a DVD, a magnetic tape or a USB stick, on which electronically-readable control information, in particular software, is stored. When this control information is read from the data medium and stored in a processing unit, all forms of embodiment of the method described above can be carried out. In this way, an embodiment of the invention can also be based on the computer-readable medium and/or the computer-readable storage medium. The advantages of the proposed computer program products or of the associated computer-readable media essentially correspond to the advantages of the proposed method.

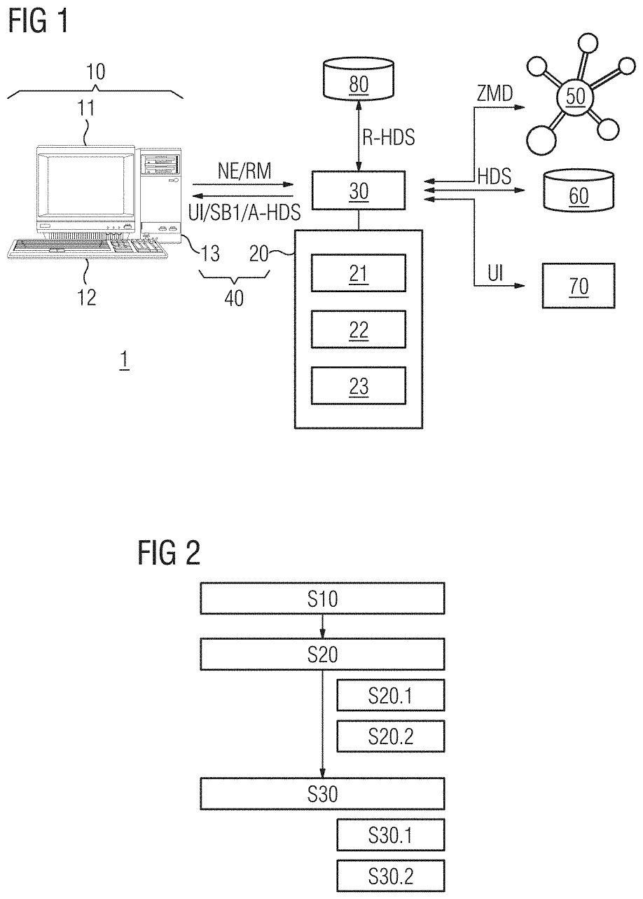

Shown in FIG. 1 is a system 1 for provision of examination information UI based on a histopathology dataset HDS in accordance with a form of embodiment. The system 1 has a user interface 10, a processing unit 20, and an interface 30 as well as, optionally, a memory unit 60, a histopathological analysis system 70 and a database 80. The system can further be connected via the interface 30 to a medical information system 50 and/or have such a system. The processing unit 20 is basically embodied for computation and provision of examination information UI based on a histopathology dataset HDS and if necessary medical data ZMD assigned to the histopathology dataset HDS. The histopathology dataset HDS, for which the examination information UI is to be established, is also called the histopathology dataset HDS to be appraised. The histopathology dataset HDS can be provided to the processing unit 20 via the interface 30 by the memory unit 60. The assigned medical data ZMD can be retrieved from the memory unit 60, the database 80 and/or the medical information system 50.

The memory unit 60 can be embodied as a central or local database. The memory unit 60 can in particular be part of a server system. The memory unit 60 is in particular embodied to store one or more histopathology dataset HDS and make it available to the processing unit 20 on request. The database 80 can be embodied as a central or local database. The database 80 can in particular be part of a server system. The database 80 is in particular embodied to store a number of reference histopathology datasets R-HDS and provide them to the processing unit 20 for a comparison with the histopathology dataset HDS to be appraised. The reference histopathology datasets R-HDS can be seen as a form of medical data ZMD assigned to the histopathology dataset HDS to be appraised. Both the memory unit 60 and also the database 80 can in particular be part of the medical information system 70.

The medical information system 70 can for example comprise a Hospital Information System (or HIS for short) a Laboratory Information System (LIS) a Radiology Information System (RIS), a Cardiovascular Information System (or CVIS for short) a Picture Archiving and Communicating System (PACS) and/or combinations of the above-mentioned systems. The assigned medical data ZMD able to be retrieved from the medical information system 70 can accordingly for example be laboratory and/or radiology data, one or more earlier appraisals for the patient, patient information (for example relating to the age, the gender and/or the previous illnesses of the patient) and the like. The assigned medical data ZMD can further comprise user information (for example relating to a name of a user and/or one or more earlier user treatments and/or its stored preferences for further analysis steps). The assigned medical data ZMD can further comprise one or more electronic medical textbooks or compendia. What is more, similar old cases of comparable patients (such as in the form of one or more reference histopathology datasets R-HDS) can be included in the assigned medical data ZMD. One or more guidelines relevant for the diagnosis of the patient can further be included in the assigned medical data ZMD.

The histopathology datasets HDS are each uniquely assigned or able to be uniquely assigned to a patient. A histopathology dataset HDS in particular has one or more histopathology slide images. Preferably all histopathology slide images of a histopathology dataset HDS are based on a single tissue sample of a patient.

The tissue sample has been taken from the patient from an anatomical target region. The anatomical target region can for example be an organ or a tissue region, which have been identified for example with an imaging modality such as an MR or CT device. The tissue sample can have been taken from the patient for example in the course of a biopsy, an operation as operation preparate or excision. Micrometer-thick tissue slides are created from the tissue samples. Typically a number of regions (known as punch biopsies or blocks are punched from a tissue sample with a punch cylinder, which are then cut into thin slices. The tissue slides arising can be fixed, prepared and readied by different techniques before they are finally stained by histopathological staining. Histological stains serve on the one hand to increase the contrast of the tissue and cell structures contained in the slides. On the other hand histological stains can be explicitly employed to highlight specific features and thus to address specific pathological issues. There are a plurality of different histological stainings, which have been developed in the course of the last 120 years. In first place there is mostly hematoxylin-eosin staining (H&E staining) as routine and overview staining. Based on the results with such a first histopathological staining, second further histopathological stainings—also called special stainings—are then ordered.

In modern laboratories computer-controlled automatic staining equipment is mostly used, at least for widely-used stainings, or further stainings can be ordered via an electronic ordering system. These components can be part of the, in particular automated, histopathological analysis system 70 in the system 1 for example.

The stained tissue slides are subsequently digitized. Specialized scanners, known as slide scanners, are used for this, which can likewise be part of the histopathological analysis system 70. The image recorded in such cases is also referred to as a “whole slide image”. The image data recorded in this process is typically two-dimensional pixel data, wherein each pixel is assigned a color value.

The histopathology datasets HDS in such cases have at least one first histopathology slide image SB1, which was created using a first histopathological staining. Preferably this first histopathological staining is an overview staining, which is used as a starting point for deciding about the further histopathological examination of the case to be appraised. In particular this first histopathological staining can comprise an H&E staining.

In addition a histopathology dataset HDS can have non-image data or metadata. The metadata can overlap entirely or partly with the assigned medical data ZMD. For example the metadata can likewise have patient information, user information and/or one or more earlier appraisals. In addition the metadata can comprise tissue sample information. The tissue sample information can for example comprise an origin of the tissue sample or a location at which the tissue sample was taken. The tissue sample information can further comprise one or more specifications about the position and type of the punch biopsies taken from the tissue sample. The tissue sample information can comprise one or more specifications about the position and type of the tissue slides produced or possible. In addition the tissue information can contain a specification about the condition of the tissue, e.g. in the form of a macroscopy report.

The user interface 10 has a display unit 11 and an input unit 12. The user interface 10 can be embodied as a portable computer system, such as possibly a smartphone or a tablet computer. The user interface 10 can further be embodied as a desktop PC or laptop. The input unit 12 can be integrated into the display unit 11, for example in the form of a touch-sensitive screen. As an alternative or in addition the input unit 12 can have a keyboard or a computer mouse and/or a digital stylus. The display unit 11 is embodied to display individual or a number of histopathology slide images SB1, SB2 and/or the examination information UI established. The user interface 10 is further embodied to receive from the user a user entry NE relating to the establishment of the examination information UI and/or an acknowledgement RM of a user relating to examination information UI established.

The user interface 10 has one or more processors 13, which are embodied to execute software for activation of the display unit 11 and the input unit 12, in order to provide a graphical user interface, which makes it possible for the user to view one or more histopathology slide images SB1, SB2, to apply one or more analysis tools to the histopathology slide images SB1, SB2, to analyze the examination information UI established and input user entries NE and/or acknowledgements RM in response. The user can activate the software for example via the user interface 10, by downloading it from an App store for example. In accordance with further forms of embodiment the software can also be a client-server computer program, in the form of a Web application, which runs in a browser.

The interface 30 can have one or more individual data interfaces, which guarantee the exchange of data between the components 10, 20, 50, 60, 70, 80 of the system 1. The one or more data interfaces can be part of the user interface 10, the processing unit 20 of the medical Information system 50, the memory unit 60, the histopathological analysis system 70 and/or the database 80. The one or more data interfaces can have a hardware and/or software interface, e.g. a PCI bus, a USB interface, a FireWire interface, a ZigBee or a Bluetooth interface. The one or more data interfaces can have an interface of a communication network, wherein the communication network can have a Local Area Network (LAN), for example an Intranet or a Wide Area Network (WAN). Accordingly the one or more data interfaces can have a LAN interface or a Wireless LAN interface (WLAN or Wi-Fi).

The processing unit 20 can have a processor. The processor can have a Central Processing Unit (CPU), Graphics Processing Unit (GPU), Digital Signal Processor (DSP), image processing processor, integrated (digital or analog) circuit or combination of the aforementioned components and further facilities for processing of histopathology image data according to forms of embodiment of the invention. The processing unit 20 can be implemented as a single component or can have one of more components, which operate in parallel or serially. As an alternative the processing unit 20 can have a real or virtual group of processors, such as maybe a cluster or a Cloud. Such a system can be called a server system. The processing unit 20 can further have a main memory, such as RAM, in order for example to store the histopathology datasets HDS and/or individual histopathology slide images SB1, SB2. As an alternative such a main memory can also be arranged in the user interface 10. The processing unit 20 is embodied by design or hardware for example by computer-readable instructions, so that it can carry out one or more method steps in accordance with forms of embodiment of the present invention. The processing unit 20 can in particular be embodied to execute an analysis algorithm for establishing the examination information UI.

The processor unit 20 can have subunits or modules 21-23, which are embodied to make available examination information UI to a user as part of an ongoing human-machine interaction and in this way support them in the appraisal of the histopathology dataset HDS to be appraised.

The module 21 is a data provision module, which is embodied to provide the histopathology dataset HDS to be appraised, and also where necessary assigned medical data ZMD. For example the module 21 can be embodied to receive from memory unit 60 the histopathology dataset HDS to be appraised and load it into the processing unit 20 or the user interface 10. This can for example be done in response to a command of the user entered via the user interface 10 or initiated automatically. The module 21 can further be embodied, in response to a command or automatically, to display to the user via the user interface 10 individual histopathology slide images SB1, SB2 of the histopathology dataset HDS to be appraised. The module 21 can further be embodied automatically to retrieve the assigned medical data ZMD from the medical information system 50, the memory unit 60 and/or the database 80.

The module 22 is an analysis module, which is embodied to establish the examination information UI. To this end the module 22 is embodied to evaluate the histopathology dataset HDS and here in particular the first histopathology slide images SB1. The module 22 can further be embodied, during the establishment of the examination information UI, to take account of any metadata in the histopathology dataset HDS and/or assigned medical data ZMD. The module 22 can further be embodied to take account of a user entry NE or an acknowledgement RM about examination information UI during the establishment of the examination information UI.

Module 23 can be construed as a dialog module, which is embodied to display the examination information UI to the user via the user interface 10 and to receive the user entry NE or the acknowledgement RM from the user.

The division of the processing unit 20 into elements 21-23 merely serves in this case to simplify the explanation of the way in which the processing unit 20 functions and is not to be understood as restrictive. The elements 21-23 or their functions can also be grouped together into one element. The elements 21-23 can also be construed in this case in particular as computer program products or computer program segments that, when executed in the processing unit 20, realize one or more of the method steps described below.

The processing unit 20 and the processor 13 can together form the controller 40. It is pointed out that the layout of the controller 40 shown, i.e. the division into the processing unit 20 and the processor 13 depicted, is likewise only to be understood as being by way of example. The processing unit 20 can thus be fully integrated into the processor 13 and vice versa. In particular the method steps can run entirely on the processor 13 of the user interface 10 by executing a corresponding computer program product (e.g. software installed on the user interface 10), which then interacts directly with the memory unit 60 via the interface 30. In other words the processing unit 20 would then be identical to the processor 13.

As already mentioned the processing unit 20, in accordance with a few forms of embodiment, can alternatively be construed as a server system, such as e.g. a local server or a Cloud server. With an embodiment of this type the user interface 10 can be referred to as “frontend” or “client”, while the processing unit 20 can then be construed as “backend”. Communication between the user interface 10 and the processing unit 20 can then be carried out for example based on an https protocol. The processing power in such systems can be divided between the client and the server. In a “thin client” system the server has the greater part of the processing power available to it, while the client in a “thick client” system provides more processing power. The same applies for the data (here: in particular the histopathology dataset HDS). While in a “thin client” system the data remains mostly on the server and only the results are transferred to the client, data is also transferred to the client in a “thick client” system.

Shown in FIG. 2 is a schematic flow diagram of a method for establishment of and provision of examination information UI based on a histopathology dataset HDS to be appraised. The examination information UI is directed in this case to a further histopathological analysis or examination, building on first histopathology slide images SB1 already present. The sequence of the method steps is not restricted either by the order shown or by the numbering chosen. This means that where necessary the sequence of the steps can be changed and individual steps can be left out.

A first step S10 is directed to the provision of the histopathology dataset HDS. The provision can be realized in this case by a retrieval of the histopathology dataset HDS from the memory unit 60 and/or a loading of the histopathology dataset HDS into the processing unit 20. The histopathology dataset HDS contains histopathological data of a patient to be appraised. In particular the histopathology dataset HDS comprises one or more first histopathology slide images SB1. The first histopathology slide images SB1 have been created based on a tissue sample of the patient. To create the one or more first histopathology slide images SB1 one or more tissue slides were prepared accordingly from the tissue sample. The one or more first histopathology slide images SB1 show tissue slides that have been stained with the same first histopathological staining. The first histopathology slide images SB1 can indicate one or more histopathological appraisals to the user making the appraisal, which are to be confirmed or discarded with one or more further histopathological analyses or examinations. In such cases there are typically a plurality of possible options for the user making the appraisal.

There is therefore provision in step S20, based on the information available, to limit the possible further histopathological examinations automatically. For the present case, in step S20 in particular promising and/or recommended and/or even prescribed further examinations are established. The result of this automatic analysis is provided to the user as examination information UI. The examination information UI can in other words have a recommendation in respect of a further histopathological examination. In particular the examination information UI can specify which further histopathological stainings (called “second histopathological stainings” below) are suitable for further diagnosis and which of the available tissue slides from the tissue sample of the patient are suitable for a staining with the one or more second histopathological stainings. The examination information UI can in other words have a specification, which is directed to the creation of second histopathology slide images SB2 from the tissue sample of the patient.

As an alternative or in addition the examination information UI can furthermore specify whether a consultation with a further pathology expert or an expert outside the specialist field of pathology is indicated. The examination information UI can further specify whether a further tissue sample of the patient is to be taken and, optionally, which parameters are to be taken into account for the corresponding tissue removal. The examination information UI can further contain a specification about whether a molecular pathological analysis is indicated and how suitable parameters of such a molecular pathological analysis can look. A molecular pathological analysis in this case can comprise an examination of a tissue sample of a patient with gene analysis methods/systems. For example a molecular pathological analysis can comprise a sequencing of a gene sequence or a determination of one or more gene expression levels. For this for example known techniques such as genotyping, microarray, Polymerase Chain Reaction (PCR), Copy-Number Variation (CNV), or what is known as (whole) genome sequencing techniques can be used.

As an alternative or in addition the examination information UI can comprise one or more confidence values, with the confidence values representing a measure of how reliable and/or unique the specifications contained in the examination information UI are.

The information available for the derivation of the examination information UI naturally just comprises histopathology dataset HDS itself. Furthermore the information available can comprise medical data ZMD assigned to the histopathology dataset HDS or to the patient.

In respect of the histopathology dataset HDS in particular the histopathology slide images SB1 already contained therein can be evaluated. In this way the analysis of the first histopathology slide images SB1 can not only provide information about a further second histopathological staining but can also disclose which tissue slides from the tissue sample are suitable for a further analysis. For this for example the tissue structure and/or a tissue density and/or a cell density and/or a proportion of tumor cells of the first histopathology slide images SB1 can be evaluated. A cell density, a cell morphology, a distribution of a histopathological staining, an (average) cell size and the like can be evaluated.

Non-image data of the histopathology dataset HDS can also further be evaluated for establishing the examination information UI (where present). This non-image data or metadata of the histopathology dataset HDS can for example comprise specifications relating to the tissue samples (tissue sample information) of the patient (such as the removal location and/or the punch biopsies and/or tissue slides available). The non-image data can further comprise patient information, such as for example relating to the age, the gender, previous illnesses or lifestyles of the patient.

Where additionally assigned medical data ZMD is to be taken into account, in an optional step S20.1 there can be provision for providing or retrieving such data. To this end for example the medical information network 50 can be interrogated for assigned medical data ZMD. For example the electronic patient records of the patient can be retrieved for this purpose and searched for patient information and/or user information for example.

In order to arrange the automatically created establishment results more comprehensibly for the user making the appraisal, the examination information UI can optionally comprise one or more hints about the basis on which (or on which data or information basis) the specification contained in the examination information UI were obtained and which sources were weighted in which way. For example it can be specified in this context for a proposed second histopathological staining that this is indicated on account of a clinical guideline or is indicated by patient information.

In accordance with a few forms of embodiment there is provision in particular for applying a suitable analysis algorithm to the histopathology dataset HDS or generally to the information available. The analysis algorithm in this case is embodied, based on the histopathology dataset HDS and where necessary on the assigned medical data ZMD, to determine the examination information UI. The analysis algorithm can further be embodied to interrogate the medical information network 50 and/or the memory unit 60 and/or the database 80 for assigned medical data ZMD. The analysis function can be understood in particular as a computer program product, of which the program elements can cause the system 1 to carry out one or more of the steps described herein. An optional step S20.2 is directed in this context to the provision of the analysis function. The provision of the analysis function can be realized in this case by retrieving the analysis function from a given memory unit and/or loading the analysis function into the processing unit 20.

In a further step S30 the examination information UI is finally provided. Provided can mean in general that the examination information UI is made available for a user. For example the examination information UI or parts of the examination information UI can be displayed to a user via the user interface 10 (step S30.1). In addition or as an alternative the examination information UI can be transferred to the histopathological analysis system 70 and here in particular to a robot staining system and/or to an electronic ordering system (step S30.2).

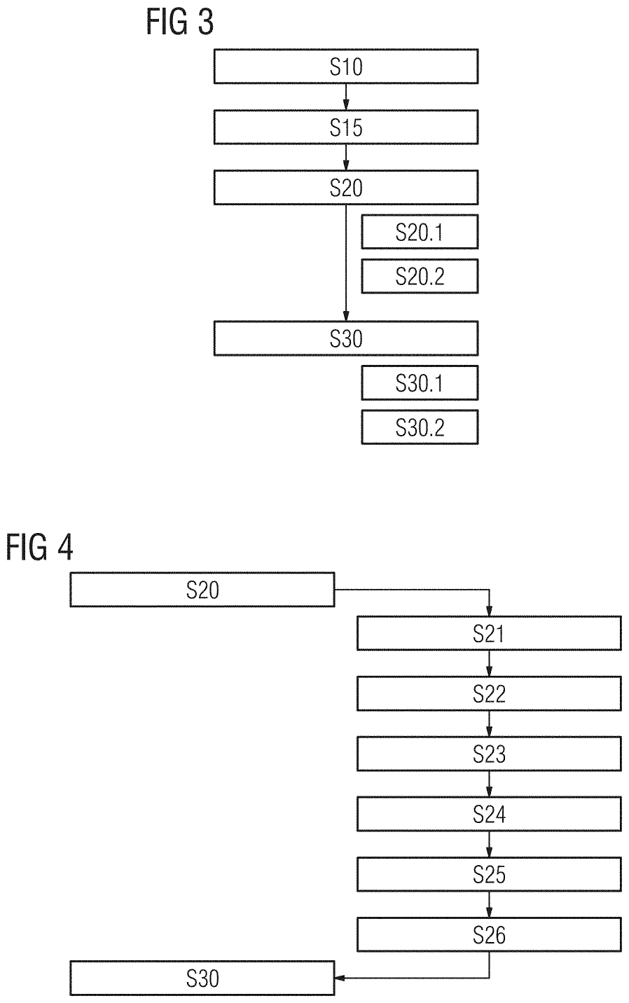

Shown in FIG. 3 is a schematic flow diagram of a method for establishment and provision of examination information UI in accordance with a further form of embodiment. The sequence of the method steps is not restricted either by the order shown or by the numbering chosen. This means that where necessary the sequence of the steps can be changed and individual steps can be left out.

The execution sequence shown in FIG. 3 differs from the sequence shown in FIG. 2 in that, before the establishment of the examination information in step S20, in step 15 a user entry NE is received via the user interface from the user making the appraisal. The user entry NE is then taken into account in step S20 in the establishment of the examination information UI. This enables the user to influence the examination information UI with a user entry NE. In accordance with one form of embodiment the user can enter one or more suspected diagnoses in the user entry NE for example, to which the examination information UI can be tailored in step S20. In this way second histopathological stainings can be explicitly proposed in the examination information UI for example, which are suitable for firming up or refuting the suspected diagnoses of the user making the appraisal. In accordance with a further form of embodiment the user making the appraisal can already specify a desired second histopathological staining in the user entry NE. The examination information UI can then designate one or more tissue slides from the tissue sample that are especially suitable for further analysis with the second histopathological staining.

Shown in FIG. 4 are optional substeps of step S20, which schematically represent an establishment of the examination information UI taking into account similar patients or similar cases in accordance with one form of embodiment. The sequence of the method steps is not restricted either by the order shown or by the numbering chosen. This means that where necessary the sequence of the steps can be changed and individual steps can be left out.

A first step S21 of such a similarity analysis is directed to an extraction of a feature signature based on the histopathology dataset HDS and/or on the medical data ZMD assigned to the histopathology dataset HDS. The feature signature can have a number of individual features that have been extracted from the histopathology dataset HDS and/or from the assigned medical data ZMD and which, as a whole, characterize the patient to be appraised—at least for histopathological issues. The feature signature can have what is known as a feature vector, in which individual features are grouped together. The features can for example comprise a pattern, a texture and/or a structure from the one or more first histopathology slide images SB1. Furthermore the features of the feature signature can have parameters that identify the (cell) density, the proportion of tumor cells and/or the density of the first histopathological staining. One or more features of the feature signature can further have parameters that designate a color value, a grey scale or a contrast value in the first histopathology slide image SB1. In addition one or more features of the feature signature can be directed to characteristics that lie outside the first histopathology slide images SB1. For example these can be features obtained from the patient and/or user information.

In a next step S22 reference datasets are provided, with which the present case to be appraised is to be compared in order to find similar cases. The reference datasets can each have a reference histopathology dataset R-HDS and/or the medical data ZMD assigned to the reference histopathology dataset R-HDS. The reference datasets can in particular be characterized in that any further histopathological examinations have already been carried out in these cases and are thus known. The reference histopathology datasets R-HDS can essentially have the same format as the histopathology dataset HDS, i.e. can contain one or more histopathology slide images SB1, SB2 and where necessary additional non-image data. The reference datasets can be provided for example by the database 80, in which at least the reference histopathology datasets R-HDS are stored. Should medical data ZMD assigned to the reference histopathology datasets R-HDS be needed for the similarity analysis, the data can be retrieved via the medical information network 50 for example. As an alternative the medical data ZMD assigned to the reference histopathology datasets R-HDS can be stored in the database 80.

A next step S23 is directed to the provision of feature signatures for the reference datasets. These can either already be present in the database 80 or the reference histopathology datasets R-HDS, or can be extracted in step S23.

In a next step S24 the feature signature of the present case to be appraised is compared with the corresponding feature signatures of the reference datasets. In this case a similarity metric can in particular be determined for each reference dataset, with the similarity metric representing a measure for a similarity or a match between the feature signature determined for the case to be appraised and the respective feature signature of the respective reference dataset. For example the similarity metric can be defined as a distance between the feature signatures in the feature space. In this way for example a simple search can be made for all patients within a specific age window with the same gender. For more complex issues individual features can be weighted differently.

In step S25, based on the comparison from the reference datasets, the similar reference datasets and in particular the similar histopathology datasets A-HDS are selected. Optionally these similar histopathology datasets A-HDS can be displayed to the user via the user interface 10.

Then, in step S26, based on the identified similar reference datasets A-HDS, the examination information UI is computed. In particular the known further histopathological examinations from the similar reference datasets A-HDS carried out can be evaluated for this.

Shown in FIG. 5 are optional steps for an ongoing human-machine interaction for optimization or adaptation of the examination information UI, starting from step S30 in accordance with one form of embodiment. The sequence of the method steps is not restricted either by the order shown or by the numbering chosen. This means that where necessary the sequence of the steps can be changed and individual steps can be left out.

Starting from the display of the examination information UI via the user interface 10 in step S30.1, in step S40 an acknowledgement RM of the user relating to the examination information UI is received via the user interface 10. The acknowledgement RM can for example have a confirmation or correction of the specifications contained in the examination information UI. The acknowledgement RM can further comprise a selection of one or more options directed to the creation of second histopathology slide images SB2. For example the user can select one or more second histopathological stainings via the acknowledgement RM.

In a step S50 the acknowledgement can be used for an adaptation of the examination information UI. This can comprise a simple correction of the examination information UI, if certain options are discarded for example. Optionally however this can also include at least parts of the examination information UI being newly established. In other words step S20 can then be executed again based upon the acknowledgement RM. In this optional case the acknowledgement RM can be treated as a user entry NE and be supplied for processing to step S15 (cf. FIG. 3).

The examination information UI adapted in step S50 can optionally be transferred to the histopathological analysis system 70—and here in particular to a robot staining system and/or an electronic ordering system (step S30.2).

In a step S60 the acknowledgement RM can further be provided to the analysis algorithm. This enables the feedback of the user to be directly accessed during use in the field, through which the analysis algorithm can be continuously improved.

The analysis algorithm can have one or more trained functions. These can for example be embodied to classify the case to be appraised based on the histopathology dataset and where necessary on the assigned medical data ZMD and in this way to identify one or more further histopathological examinations. In other words the analysis algorithm can comprise one or more electronic classification algorithms, which can be trained in particular by machine learning. In particular support vector machine algorithms, decision tree algorithms, k-nearest neighbors algorithms, Bayes classification algorithms, (convolutional) neural networks and/or combinations hereof can be used.

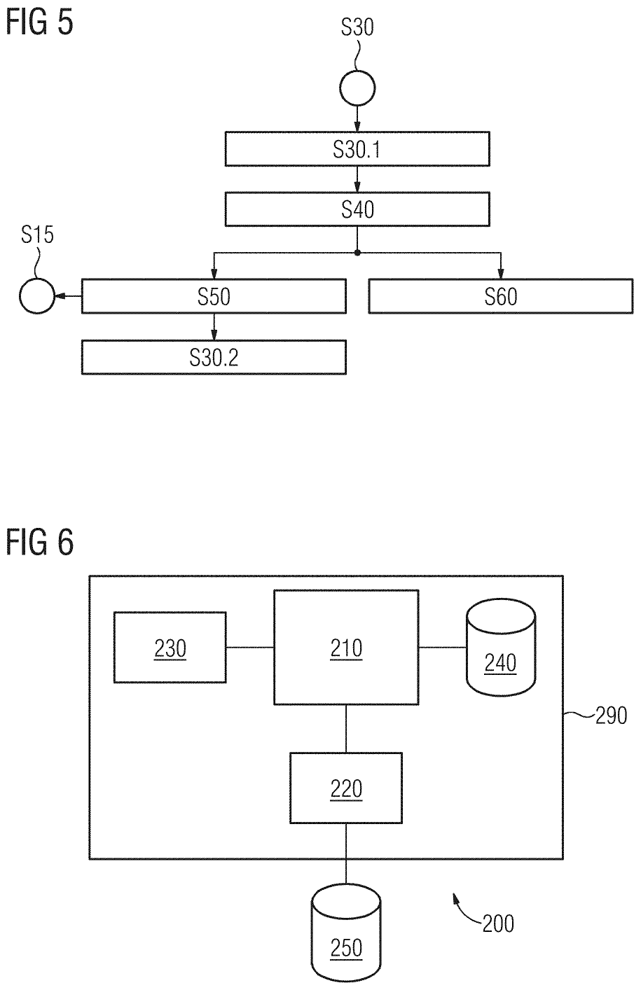

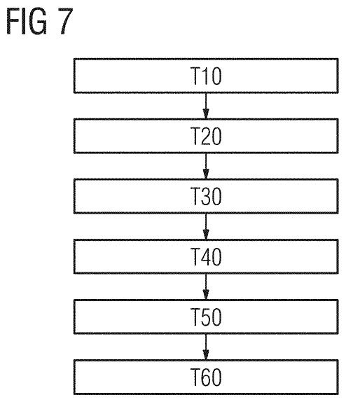

FIG. 6 shows a form of embodiment of a system 200 for training or provision of the analysis algorithm. The system comprises a processor 210, an interface 220, a main memory 230, a memory facility 240 and a database 250. The processor 210, the interface 220, the main memory 230 and the memory facility 240 can be embodied as a computer 290. The processor 210 controls the operation of the computer 290 during the training of the analysis algorithm. In particular the processor 210 can be embodied in such a way that it carries out the method steps shown in FIG. 7. Corresponding instructions can be stored in the main memory 230 or in the memory facility 240 and/or loaded into the main memory 230 when execution of the instructions is required. The memory facility 240 can be embodied as a local memory or as a remote memory to which there is access via a network. The method steps shown in FIG. 7 can be defined by computer program products, which are stored in the main memory 230 and/or the memory facility 240.

The database 250 can be implemented as Cloud memory or local memory that has a connection to the computer 290 via the wireless or wired interface 220. The database 250 can in particular also be part of the computer 290. The database 250 serves as an archive for the training histopathology dataset HDS T-HDS and/or the medical data ZMD assigned to the training histopathology dataset HDS T-HDS. The database 250 can further serve as an archive for one or more trained analysis algorithms.

Shown in FIG. 7 is a schematic flow diagram of a method for provision of an analysis algorithm for establishment of examination information UI relating to a further histopathological examination based on a histopathology dataset HDS. The sequence of the method steps is not restricted either by the order shown or by the numbering chosen. This means that where necessary the sequence of the steps can be changed and individual steps can be left out.

A first step T10 is directed to the provision of an analysis algorithm. The analysis algorithm can in particular have one or more trained functions. In this case the analysis algorithm can be provided to the processor 210 via the interface 220 from the database 250. The analysis algorithm in this case can already be pretrained, i.e. one or more parameters of the trained function(s) contained therein can have been adapted by the described training method and/or another training method. As an alternative the one or more parameters of the trained function(s) contained therein can have been not yet adapted via training data, in particular the one or more parameters can be preset to a constant value and/or to a random value. In particular all parameters of the trained function(s) contained therein can have not yet been adapted via training data, in particular all parameters can have been preset to a constant value and/or by a random value.

A second step T20 is directed to the provision of training input data. Since, when used, the analysis algorithm is to determine one or more further histopathological examinations based on existing histopathological information for a patient, suitable training input data is histopathology datasets HDS and where necessary medical data ZMD (outside the histopathology datasets) assigned to these. Histopathology datasets will be called training histopathology datasets T-HDS below. The training histopathology datasets T-HDS can basically have the same format or the same configuration as the histopathology dataset HDS. In particular the training histopathology datasets T-HDS contain one or more first histopathology slide images SB1. The provision of a training histopathology dataset T-HDS to processor 210 can be done by retrieving it from the database 250 via the interface 220. The optional provision of assigned medical data ZMD to the retrieved training histopathology dataset T-HDS can likewise be implemented by retrieving it from the database 250 or by interrogating the medical information system 70 optionally connected via the interface 220 to the processor 210.

Step T30 is directed to the provision of training output data. The training output data in this case is verified examination information UI. The verified examination information UI in this case represents target values, which specify to the analysis algorithm how a suitable examination information UI for the respective training histopathology dataset T-HDS can look. The verified examination information UI in this case can have in each case one or more specifications about the further histopathological examinations that are to be carried out or have been carried out for the respective training histopathology dataset T-HDS and/or whether these are expedient. For example verified examination information UI can specify which second histopathological stainings and which tissue slides have been used in each case starting from the respective first histopathology slide images SB1 in order to create one or more further second histopathology slide image SB2. The verified examination information UI can be based in particular on an annotation by a user. The provision of the verified examination information UI to the processor 210 can be done by retrieving it from the database 250 via the interface 220.

In a next step T40 the training input data, i.e. the training histopathology datasets R-HDS and also where necessary the assigned medical data ZMD are input into the analysis algorithm. On this basis the analysis algorithm computes examination information UI, which is to contain one or more specifications about further histopathological examinations based on the information available.

In a next step T50 the examination information UI established in this way is compared with the corresponding verified examination information UI. Then, based upon this comparison, the analysis algorithm can be adapted in step T60. This can be done for example based upon a cost functional that penalizes deviations of the examination information UI established from the corresponding verified examination information UI. One or more parameters of the trained function(s) contained in the analysis function can then in particular be adapted so that the cost functional is minimized, for example via a back propagation. For minimization of the cost functional a comparison is carried out for different paired sets consisting of established and verified examination information UI, until a local minimum of the cost functional is reached and the trained function works satisfactorily.

Where this has not happened explicitly, but is sensible and in the spirit of the invention, individual example embodiments, individual or their subaspects or features can be combined with one another or exchanged, without departing from the framework of the current invention. Advantages of the invention described with regard to one example embodiment also apply, without this being explicitly stated, where they are able to be transferred, to other example embodiments.

Even if not explicitly stated, individual example embodiments, or individual sub-aspects or features of these example embodiments, can be combined with, or substituted for, one other, if this is practical and within the meaning of the invention, without departing from the present invention. Without being stated explicitly, advantages of the invention that are described with reference to one example embodiment also apply to other example embodiments, where transferable.

Of course, the embodiments of the method according to the invention and the imaging apparatus according to the invention described here should be understood as being example. Therefore, individual embodiments may be expanded by features of other embodiments. In particular, the sequence of the method steps of the method according to the invention should be understood as being example. The individual steps can also be performed in a different order or overlap partially or completely in terms of time.

The patent claims of the application are formulation proposals without prejudice for obtaining more extensive patent protection. The applicant reserves the right to claim even further combinations of features previously disclosed only in the description and/or drawings.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

None of the elements recited in the claims are intended to be a means-plus-function element within the meaning of 35 U.S.C. § 112(f) unless an element is expressly recited using the phrase “means for” or, in the case of a method claim, using the phrases “operation for” or “step for.”

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A computer-implemented method for provision of examination information relating to a further pathological examination, comprising:
provisioning a histopathology dataset, including one or more first histopathology slide images, each first histopathology slide image of the first histopathology slide images showing a first tissue slide prepared from a tissue sample of a patient and stained with a first histopathological staining;
establishing, using an analysis algorithm based on the histopathology dataset, examination information relating to a further pathological examination, the examination information including a specification for carrying out a further pathological examination of the patient; and
provisioning the examination information established.

2. The method of claim 1, wherein the examination information includes a specification for creation of one or more second histopathology slide images, the one or more second histopathology slide images being different from the one or more first histopathology slide images.

3. The method of claim 2,
wherein the examination information includes the specification, in respect of one or more further histopathological stainings; and
wherein the one or more further histopathological stainings are different from the first histopathological staining and are suitable for creation of the second histopathology slide images.

4. The method of claim 3, wherein the examination information includes the specification, in respect of one or more second tissue slides prepared or prepareable from the tissue sample, the second tissue slides being different from the first tissue slides and being suitable for creation of the second histopathology slide images.

5. The method of claim 4, wherein the examination information includes the specification of second tissue slides, each suitable for each further histopathological staining.

6. The method of claim 1, wherein:
the histopathology dataset includes tissue sample information; and
the establishing additionally occurs based on the tissue sample information.

7. The method of claim 1, further comprising:
retrieving medical data assigned to the histopathology dataset, wherein establishing additionally occurs based on the assigned medical data.

8. The method of claim 7, wherein the assigned medical data includes one or more of:
one or more items of personal data of the patient,
one or more laboratory values of the patient,
one or more items of radiology data of the patient,
one or more medical guidelines,
one or more, in particular radiological, appraisals of the patient, and
one or more earlier histopathological examination results of the patient.

9. The method of claim 1, further comprising:
selecting one or more similar histopathology datasets from a series of reference histopathology datasets, the similar histopathology datasets including a defined similarity to the histopathology dataset and for which further pathological examinations carried out are previously known, wherein the establishing is additionally performed based on the similar histopathology datasets.

10. The method of claim 1, wherein the analysis algorithm includes a trained function, the trained function being embodied, based on the histopathology dataset, to provide the examination information.

11. The method of claim 1, wherein the provisioning of the examination information comprises:
outputting of the examination information to a user making the appraisal, via a user interface.

12. The method of claim 1, further comprising:
receiving an acknowledgement relating to the examination information; and at least one of
adapting the examination information based on the acknowledgement; and
provisioning the acknowledgement to the analysis algorithm for adaptation of the analysis algorithm.

13. The method of claim 1, further comprising:
receiving a user entry, of a user making the appraisal via a user interface, in respect of a further pathological examination to be carried out,
wherein the establishing of the examination information is additionally performed based on the user entry.

14. A system for establishment of examination information relating to a further pathological examination, comprising:
an interface embodied to receive a histopathology dataset, the histopathology dataset including one or more first histopathology slide images, each first histopathology slide image of the first histopathology slide images showing a first tissue slide prepared from a tissue sample of a patient and stained with a first histopathological staining; and
a controller embodied to
establish, based on the histopathology dataset, examination information relating to a further pathological examination, the examination information including at least one specification for carrying out a further pathological examination, and
provide the examination information established.

15. A non-transitory computer program product, storing a program, directly loadable into a memory of a programmable processing unit of a controller, including program code for carrying out the method of claim 1 when the program is executed in the controller.

16. A non-transitory computer-readable memory medium, storing readable and executable program sections for carrying out the method of claim 1 when the program sections are executed by a controller.

17. The method of claim 2, wherein the examination information includes the specification, in respect of one or more second tissue slides prepared or prepareable from the tissue sample, the second tissue slides being different from the first tissue slides and being suitable for creation of the second histopathology slide images.

18. The method of claim 17, wherein the examination information includes the specification of second tissue slides, each suitable for each further histopathological staining.

19. The method of claim 3, wherein the examination information includes the specification of second tissue slides, each suitable for each further histopathological staining.

20. The method of claim 2, wherein:
the histopathology dataset includes tissue sample information; and
the establishing additionally occurs based on the tissue sample information.

21. The method of claim 3, wherein:
the histopathology dataset includes tissue sample information; and
the establishing additionally occurs based on the tissue sample information.

22. The method of claim 2, further comprising:
retrieving medical data assigned to the histopathology dataset, wherein establishing additionally occurs based on the assigned medical data.

23. The method of claim 22, wherein the assigned medical data includes one or more of:
one or more items of personal data of the patient,
one or more laboratory values of the patient,
one or more items of radiology data of the patient,
one or more medical guidelines,
one or more, in particular radiological, appraisals of the patient, and
one or more earlier histopathological examination results of the patient.

24. The method of claim 2, further comprising:
selecting one or more similar histopathology datasets from a series of reference histopathology datasets, the similar histopathology datasets including a defined similarity to the histopathology dataset and for which further pathological examinations carried out are previously known, wherein the establishing is additionally performed based on the similar histopathology datasets.

25. The method of claim 10, wherein the trained function in is embodied to evaluate the one or more first histopathology slide images.

26. The method of claim 2, further comprising:
receiving an acknowledgement relating to the examination information; and at least one of
adapting the examination information based on the acknowledgement; and
provisioning the acknowledgement to the analysis algorithm for adaptation of the analysis algorithm.
